(19) United States
(12) Patent Application Publication
(10) Pub. No.: US 2010/0161021 A1
(43) Pub. Date: Jun. 24, 2010

(54) MYOCARDIAL PAD

(75) Inventors: Kenji Iino, Kanazawa-shi (JP); Go Watanabe, Kanazawa-shi (JP); Nobuhiko Yui, Nomi-shi (JP); Tooru Ooya, Nomi-shi (JP); Ryoji Kawabata, Nomi-shi (JP)

Correspondence Address: BIRCH STEWART KOLASCH & BIRCH, PO BOX 747, FALLS CHURCH, VA, 22040-0747, US

(73) Assignees: National University Corporation Kanazawa University, Kanazawa-shi (JP); Japan Advanced Institute Of Science And Technology, Nomi-Shi (JP)

(21) Appl. No.: 12/663,204

(22) PCT Filed: Jun. 7, 2007
(86) PCT No.: PCT/JP2007/061963

§ 371 Date: Dec. 24, 2009

(51) Int. Cl. A61N 1/05

(52) U.S. Cl. 607/129

(57) ABSTRACT

This invention relates to a myocardial pad used in cardioversion, pacing, or the like and to a myocardial lead and a therapeutic apparatus for cardiac disease comprising the same. The myocardial pad of this invention is bondable to epicardia and has conductivity, biocompatibility, and biodegradability. Using this pad, the myocardial lead can be immobilized onto the atrium without suture. Thus, bleeding from the atrium, which is a lethal complication, caused by lead removal can be prevented.

### TECHNICAL FIELD

The present invention relates to a myocardial pad used in cardioversion, pacing, or the like and to a myocardial lead and a therapeutic apparatus for cardiac disease comprising the same.

### BACKGROUND ART

Atrial fibrillation is a complication most commonly seen after heart surgery. Even after the 50-year history of heart surgery, the frequency of occurrence thereof still stays at 10 to 40%. The time of the occurrence is statistically shown to be usually between the postoperative 2nd and 4th days. The atrial fibrillation aggravates the pumping action of the heart, resulting in 10 to 20% decrease in cardiac output. This causes, in patients with low cardiac functions, failure of hemodynamics and atrial thrombus that are involved in the development of secondary complications such as perioperative cerebral infarction and myocardial infarction, leading to longer hospitalization associated therewith and even to increase in medical expense. In Europe and America, an attempt has been made since the late 1990, which involves: placing a defibrillation lead in the epicardia of the right and left atria during heart surgery; directly defibrillating the heart at low energy at a point in time when atrial fibrillation occurs after the surgery; and transdermally removing the defibrillation lead no longer required in a stable phase after the surgery. This method achieves defibrillation at a few joules and does not necessarily require administration of sedative agents. Moreover, the method is useful in terms of immediate and reliable effects. However, the lead must be sewed over about 5 to 10 cm on both the atria, and this technique is complicated. Moreover, bleeding from the atrium, which is a lethal complication, caused by lead removal might occur. Thus, the method has still not spread.

Moreover, in pacing, a lead is often sewed directly on the cardiac muscle even today, because the lead is merely sewed at a short length on the cardiac muscle. On the other hand, a method has also been attempted, which involves performing pacing via a pad without directly sewing a pacing lead thereon.

Biocompatible materials such as collagen are conventionally used as materials for pads used in cardioversion or pacing (e.g., Patent Document 1).

However, the conventional pads exhibit no bondability to epicardia. Therefore, a myocardial lead must be sewed on the atrium as described above. Thus, these pads cannot solve the problems described above.

On the other hand, Patent Document 2 discloses that a cross-linked product obtained by cross-linking a polysaccharide having a carboxyl group and a cationic compound hardly swells after cross-linking and is useful as a material for slow release of functional substances such as drugs or as a scaffolding material for regenerative medicine. However, the document has no mention of suggesting its applicability as a myocardial pad, such as bondability to epicardia.

### Patent Document 1: JP Patent Publication (Kohyo) No. 9-508039A (1997) Patent Document 2: JP Patent Publication (Kokai) No. 2005-53974A (2005)

### DISCLOSURE OF THE INVENTION

An object of the present invention is to provide a myocardial pad that is bondable to epicardia and has conductivity, biocompatibility, and biodegradability.

The present invention is summarized as follows:

(1) A myocardial pad comprising a cross-linked product obtained by cross-linking a polysaccharide having a carboxyl group and a cationic compound.
(2) The myocardial pad according to (1), wherein the cross-linked product is a cross-linked product obtained by cross-linking the polysaccharide having a carboxyl group and the cationic compound using a condensing agent or cross-linking agent.
(3) The myocardial pad according to (2), wherein the condensing agent is water-soluble carbodiimide.
(4) The myocardial pad according to any of (1) to (3), wherein the cross-linked product is a cross-linked product obtained by freeze-drying an aqueous solution containing the polysaccharide having a carboxyl group and the cationic compound, and then cross-linking the polysaccharide having a carboxyl group and the cationic compound using a condensing agent or cross-linking agent.
(5) The myocardial pad according to any of (1) to (4), wherein the polysaccharide having a carboxyl group is glycosaminoglycan.
(6) The myocardial pad according to (5), wherein the glycosaminoglycan is hyaluronic acid.
(7) The myocardial pad according to any of (1) to (6), wherein the cationic compound is polylysine.
(8) The myocardial pad according to any of (1) to (7), wherein a molar ratio between the polysaccharide having a carboxyl group and the cationic compound is 1:9 to 1:1.
(9) The myocardial pad according to any of (1) to (8), which is used in cardioversion or pacing.
(10) A myocardial lead comprising the myocardial pad according to any of (1) to (9) and a lead having an end attached to the pad.
(11) A therapeutic apparatus for cardiac disease comprising the myocardial lead according to (10).
(12) The therapeutic apparatus for cardiac disease according to (11), which is a defibrillator or cardiac pacemaker.

The myocardial pad of the present invention is bondable to epicardia and has conductivity, biocompatibility, and biodegradability. Using this pad, the myocardial lead can be immobilized onto the atrium without suture. Thus, bleeding from the atrium, which is a lethal complication, caused by lead removal can be prevented.

### BEST MODE FOR CARRYING OUT THE INVENTION

Hereinafter, the present invention will be described in detail.

In the present invention, % refers to [weight (g)/volume (dl]×100, unless otherwise specified. Moreover, in the present invention, pure water refers to water purified by continuous ion exchange and reverse osmosis.

A polysaccharide having a carboxyl group, used in the present invention is not particularly limited and is preferably glycosaminoglycan (e.g., hyaluronic acid, chondroitin, chondroitin sulfate A, chondroitin sulfate C, chondroitin sulfate D, chondroitin sulfate E, chondroitin sulfate K, dermatan sulfate (chondroitin sulfate B), heparin, heparan sulfate, and keratan sulfate), carboxymethylcellulose, cellouronic acid, carboxymethyl chitin, or the like. These polysaccharides may be any of extracts from natural plants, microbial fermentation products, products synthesized by enzymes, and chemically synthesized products. Among them, hyaluronic acid is particularly preferable. When hyaluronic acid is used as the polysaccharide having a carboxyl group, the average molecular weight thereof is preferably at least 10 kDa, more preferably 50 kDa to 1500 kDa. In the present invention, the average molecular weight of the hyaluronic acid refers to a viscosity-average molecular weight and can be measured according to a viscosity method.

A cationic compound used in the present invention is not particularly limited as long as it has biocompatibility and biodegradability. Examples thereof include polylysine, cationic amino acids (e.g., lysine, hydroxylysine, and arginine), peptides (e.g., peptides containing lysine, hydroxylysine, or arginine as a constituent amino acid), proteins, and chitosan and preferably include polylysine. When polylysine is used as the cationic compound, the average molecular weight thereof is preferably 500 to 100 kDa, more preferably 1 k to 10 kDa. In this context, the average molecular weight of the polylysine refers to a weight-average molecular weight.

A molar ratio between the polysaccharide having a carboxyl group and the cationic compound used in the production of a cross-linked product is preferably 1:9 to 1:1, more preferably 1:5 to 1:1.5, in terms of bondability to epicardia and cytotoxicity.

The cross-linked product according to the present invention can be obtained, for example, by cross-linking the polysaccharide having a carboxyl group and the cationic compound using a condensing agent or cross-linking agent.

In the present invention, the condensing agent refers to a reagent that condenses the polysaccharide having a carboxyl group and the cationic compound without introducing a spacer therebetween. The cross-linking agent refers to a reagent that condenses the polysaccharide having a carboxyl group and the cationic compound, while introducing a spacer therebetween.

The condensing agent used in the present invention is not particularly limited, and examples thereof include water-soluble carbodiimide (WSC) and 4-(4,6-dimethoxy-1,3,5-triazin-2-yl)-4-methylmorpholinium chloride (DMT-MM). Examples of the water-soluble carbodiimide include 1-ethyl-3-(3-dimethylaminopropyl)carbodiimide (EDC) and 1-cyclohexy-3-(2-morpholinoethyl)carbodiimide metho-p-toluenesulfonate (Morpho-CDI). When water-soluble carbodiimide is used as the condensing agent, cross-linking reaction is preferably performed in a mixed solution containing the water-soluble carbodiimide, alcohol (e.g., ethanol, methanol, propanol, or isopropanol), and water.

A method for producing the cross-linked product using water-soluble carbodiimide as the condensing agent will be exemplified below.

(1) The polysaccharide having a carboxyl group and the cationic compound are dissolved in pure water to prepare a mixed aqueous solution. In this context, the concentration of the polysaccharide having a carboxyl group in the mixed aqueous solution is preferably 1 to 30%, particularly preferably 1 to 10%. Moreover, the concentration of the cationic compound in the mixed aqueous solution is preferably 0.01 to 30%, particularly preferably 0.05 to 10%.
(2) The mixed aqueous solution is poured into a mold of an arbitrary shape and freeze-dried. In this procedure, one end of a lead is placed in the mold, into which the mixed aqueous solution is in turn poured and then freeze-dried. As a result, the lead can be attached easily to a pad.
(3) This solid is dipped, for cross-linking, in an ethanol-water mixture containing the condensing agent. In this context, the concentration of the condensing agent in the ethanol-water mixture is preferably 0.1 to 10% (5 to 500 mmol/L), particularly preferably 0.5 to 5% (25 to 250 mmol/L). The ethanol concentration of the ethanol-water mixture is preferably 50 to 90% by volume, particularly preferably 70 to 85% by volume. The dipping is preferably performed at 0 to 40° C. for 5 to 50 hours. By this water mixture, the carboxyl group of the polysaccharide having a carboxyl group is activated. The activated carboxyl group is bound through an ester bond with a hydroxyl group contained in the polysaccharide, also through an ester bond with a hydroxyl group, if any, in the cationic compound, and further through an amide bond with an amino group, if any, in the cationic compound such that the polysaccharide is insolubilized to obtain a cross-linked product of the polysaccharide having the same shape and size as those of the mold. Then, the remaining water-soluble carbodiimide is washed away with water to obtain a cross-linked product of the polysaccharide.

When hyaluronic acid is used as the polysaccharide having a carboxyl group and polylysine is used as the cationic compound, first, the polylysine is dissolved at a final concentration of 0.01 to 30% in pure water while adjusted to pH 7 with an aqueous HCl solution. In the obtained aqueous polylysine solution, the hyaluronic acid is dissolved at a concentration of 1 to 30%. This aqueous solution is poured into a mold of an arbitrary shape and freeze-dried at −250 to −10° C. to obtain a white, sponge-like solid. In this procedure, one end of a lead is placed in the mold, into which the mixed aqueous solution is in turn poured and then freeze-dried. As a result, the lead can be attached easily to a pad. This solid is dipped, for cross-linking, in an ethanol-water mixture containing the condensing agent under the conditions described above to obtain a hyaluronic acid-polylysine cross-linked product.

The cross-linking agent used for cross-linking the polysaccharide having a carboxyl group and the cationic compound is not particularly limited unless the spacer portion derived from the cross-linking agent has a bad influence in vivo. Examples thereof include those having a spacer portion composed of an amino acid, a peptide, a monosaccharide, an oligosaccharide or a derivative thereof, oligo(ethylene glycol), polyethylene glycol, polyacrylic acid, polyvinyl alcohol, polyvinylpyrrolidone, or the like and having a functional group such as an epoxy, acid halide, alkyl halide, vinyl, aldehyde, methanesulfonyl, or p-toluenesulfonyl group.

When such a cross-linking agent is used, the cross-linking reaction can be performed in the same way as that using the condensing agent.

The drying of the mixed aqueous solution may be achieved by a drying method other than freeze-drying. In this case, however, it is difficult to produce a cross-linked product having the same size as that of the mold, due to a decreased size.

Moreover, the attachment of the lead to a pad can be performed easily by placing, during the freeze-drying procedure, one end of the lead in the mold, into which the mixed aqueous solution is in turn poured and then freeze-dried, as described above. This attachment can also be performed by weaving or interweaving one end of a lead (conductor) into a pad, as described in JP Patent Publication (Kohyo) No. 9-508039A (1997).

In this context, the myocardial pad of the present invention secures conductivity owing to the solvent ingredient thereof and therefore does not require causing one end of the lead to penetrate the pad such that the end is contacted with the cardiac muscle. Moreover, a structure in which the lead keeps from coming in contact with the cardiac muscle more highly improves the contact of the pad with the cardiac muscle.

A myocardial lead comprising the pad thus obtained and a lead can attached via a dedicated connector to a defibrillator, cardiac pacemaker, or the like and used as a therapeutic apparatus for cardiac disease used in cardioversion, pacing, or the like.

Moreover, the myocardial pad of the present invention can also be applied as a sensing gel that achieves direct monitoring of cardiac functions. The myocardial pad of the present invention also achieves continuous evaluation of myocardial functions (e.g., myocardial enzymatic metabolism and local myocardial oxygen consumption) directly from the surface of the heart for an arbitrary period in an arbitrary location using near-infrared spectrometry that has been studied by the present inventors since 1997, and can thus be applied as a biosensor.

### EXAMPLES

Hereinafter, the present invention will be described in detail with reference to Examples.

In Examples below, sodium hyaluronate (hereinafter, referred to as “HA”) was adopted as a polysaccharide having a carboxyl group, and ε-polylysine (hereinafter, referred to as “EPL”) was adopted as a cationic compound. HA-EPL cross-linked products (1) to (4) shown below were prepared and placed on the surfaces of the actually beating hearts of adult pigs to evaluate the bondability thereof. Methods for preparing the cross-linked products (1) and (2) will be shown specifically.

(1) 2% HA (1150 kDa):EPL=1:2 (molar ratio of constituent units)
(2) 4% HA (1150 kDa):EPL=1:2 (molar ratio of constituent units)
(3) 2% HA (1150 kDa):EPL=1:10 (molar ratio of constituent units)
(4) 10% HA (90 kDa):EPL=1:1 (molar ratio of constituent units)

### Example 1

HA (average molecular weight: 1150 kDa) is adjusted to a final concentration of 2%. 800 mg (2 mmol) of HA (average molecular weight: 1150 kDa) was dissolved in 20 ml of pure water. 512 mg (4 mmol) of EPL (average molecular weight: 3.8 kDa) was added to pure water while adjusted to pH 7 with a 1 mol/L aqueous HCl solution to bring the total amount to 20 ml. Both the solutions were mixed to prepare 40 ml of a mixed solution, to which 1760 mg of NaCl was then added. This solution was poured into a mold made of Teflon (in a disk form with a diameter of 50 mm and a depth of 5 mm), then frozen in liquid nitrogen, and then freeze-dried at −200° C. using a freeze dryer (FREEZONE 6 manufactured by Labconco Corp.). The obtained white solid was dipped at room temperature (2 to 20° C.) for 24 hours in a solution of 50 mmol/L WSC (1-ethyl-3-(3-dimethylaminopropyl)carbodiimide hydrochloride: EDC) in 80% by volume of ethanol. The remaining WSC was washed away with water (substituted by saline) to obtain an HA-EPL cross-linked product in a disk form with a diameter of 50 mm and a thickness of 5 mm.

### Example 2

HA (average molecular weight: 1150 kDa) is adjusted to a final concentration of 4%. 1600 mg (4 mmol) of HA (average molecular weight: 1150 kDa) was dissolved in 20 ml of pure water. 1024 mg (8 mmol) of EPL (average molecular weight: 3.8 kDa) was added to pure water while adjusted to pH 7 with a 1 mol/L aqueous HCl solution to bring the total amount to 20 ml. Both the solutions were mixed to prepare 40 ml of a mixed solution, to which 1760 mg of NaCl was then added. This solution was poured into a mold made of Teflon (in a disk form with a diameter of 50 mm and a depth of 5 mm), then frozen in liquid nitrogen, and then freeze-dried at −200° C. using a freeze dryer (FREEZONE 6 manufactured by Labconco Corp.). The obtained white solid was dipped at room temperature (2 to 20° C.) for 24 hours in a solution of 50 mmol/L WSC (1-ethyl-3-(3-dimethylaminopropyl)carbodiimide hydrochloride: EDC) in 80% by volume of ethanol. The remaining WSC was washed away with water (substituted by saline) to obtain an HA-EPL cross-linked product in a disk form with a diameter of 50 mm and a thickness of 5 mm.

Moreover, HA-EPL cross-linked products (3) and (4) were produced according to Examples 1 and 2.

The HA-EPL cross-linked products (1) to (4) thus obtained were placed on the right auricles and the back surfaces of the left atria of the actually beating hearts of adult pigs to study the bondability thereof. As a result, these cross-linked products followed the motions of the hearts. It was demonstrated that 2% HA (1150 kDa):EPL=1:2 (flexible) was most suitable for the curved right auricles, and 4% HA (1150 kDa):EPL=1:2 was most suitable for the less-curved left atria.

### Example 3

### Defibrillation Experiment Using Adult Pigs

Based on the results of studying the bondability, a defibrillation lead was placed as shown in FIG. 1 in a mold made of Teflon during gel preparation, and an HA-EPL solution was poured thereinto. By this method, defibrillation electrodes having 2% HA (1150 kDa):EPL=1:2 or 4% HA (1150 kDa):EPL=1:2 were prepared. The former defibrillation electrode was placed on the right auricle, and the later defibrillation electrode was placed on the back surface of the left atrium to conduct a defibrillation experiment using adult pigs.

The adult pigs were subjected to endotracheal intubation under general anesthesia. Then, their respiration was controlled using a ventilator. After thoracotomy, the hearts were exposed. Two pacing leads were indwelled in the pulmonary vein (the reference numeral 3 in FIG. 2), and the electrodes were further placed as shown in FIG. 2. Then, pacing was performed at 50 Hz for 5 minutes using a fibrillation generator (SEN-7103, Nihon Kohden Corp., Tokyo) to induce atrial fibrillation.

Successful atrial fibrillation was confirmed using an electrocardiogram. Then, the output was increased to 0.5 J, 1.0 J, 2.0 J, 3.0 J, and 4.0 J until defibrillation was achieved. A defibrillation threshold and the resistance value of the whole system were measured. Then, the two pacing leads and two defibrillation leads were removed from the bodies, and the chests were closed. On the postoperative 1st, 3rd, 5th, and 7th days of illness, atrial fibrillation was induced in the same way as above from outside the bodies without thoracotomy, and defibrillation was performed. These procedures were conducted on five adult pigs. The relationship of the postoperative day with the output and the relationship of the postoperative day with the resistance value are shown in FIGS. 3 and 4, respectively.

As a result, even on the 7th day, defibrillation was achieved. The output was relatively increased with the passage of time, and the resistance value was also increased. Nevertheless, even on the 7th day of illness, defibrillation at 2.6 J on average was achieved. The defibrillation could be conducted at energy competitive with that in conventional methods which comprise using a collagen pad or directly passing a current through the cardiac muscle. Moreover, the defibrillation could be conducted at sufficiently lower energy than that of defibrillation from outside that requires energy of 40 J or higher.

To confirm degradation, rethoracotomy was performed after the 7th day of illness. As a result, the gel did not retain the original form and assumed a membrane form that covered the electrodes and the heart. Fourteen days later, the gel was confirmed by rethoracotomy to disappear.

In conclusion, the defibrillation system using the myocardial pad of the present invention having both conductivity and degradability achieved defibrillation at a low output up to at least the 7th day of illness and was regarded as a sufficiently clinically applicable device.

### BRIEF DESCRIPTION OF THE DRAWINGS

FIG. 1 is a diagram showing a brief summary of a method for preparing a defibrillation electrode.

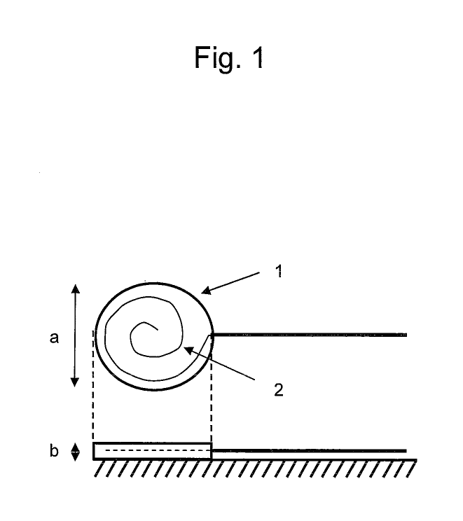

FIG. 2 is a diagram showing the placement of an electrode for the right auricle (A) and an electrode for the back surface of the left atrium (B).

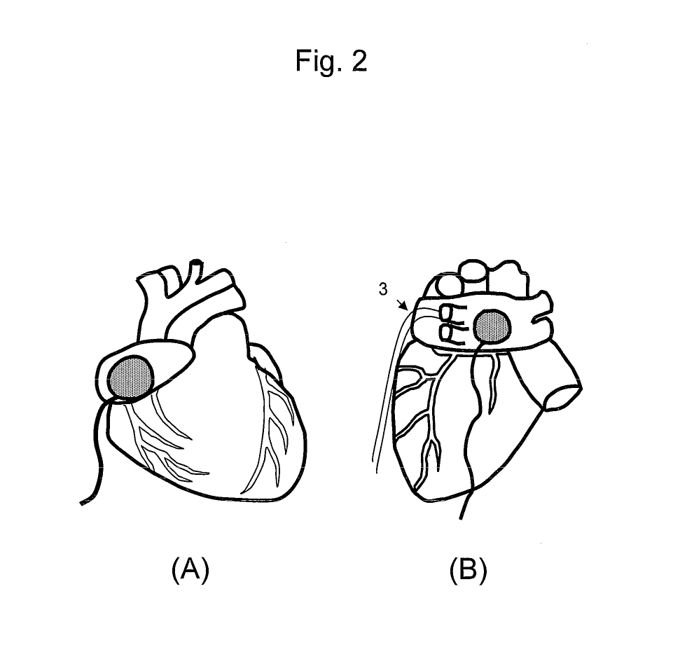

FIG. 3 is a diagram showing the relationship of a postoperative day with an output.

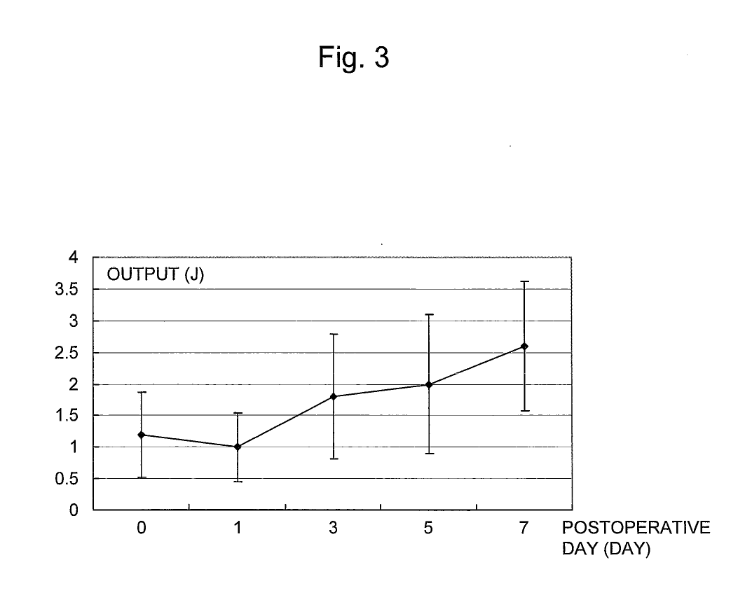

FIG. 4 is a diagram showing the relationship of a postoperative day with a resistance value.

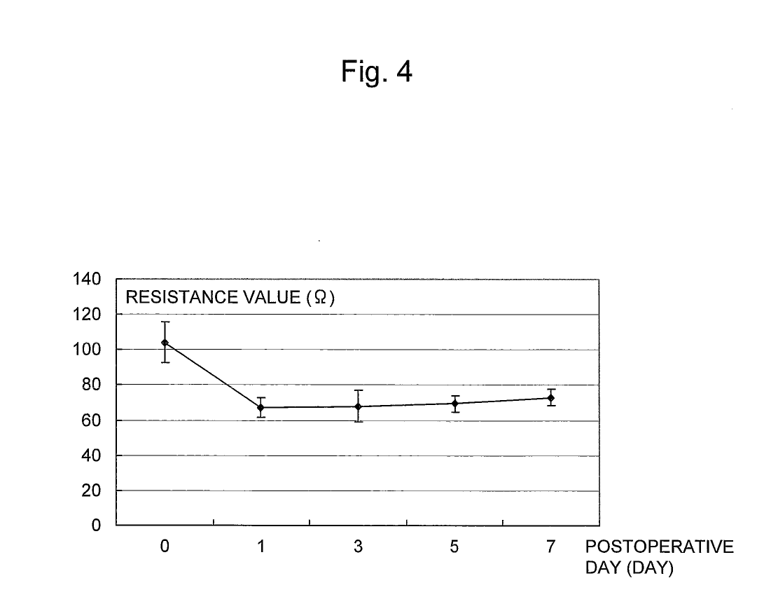

### DESCRIPTION OF SYMBOLS

1: gel pad 2: defibrillation lead 3: electrode for inducing atrial fibrillation a: mold diameter (50 mm) b: mold depth (5 mm)

1. A myocardial pad comprising a cross-linked product obtained by cross-linking a polysaccharide having a carboxyl group and a cationic compound.

2. The myocardial pad according to claim 1, wherein the cross-linked product is a cross-linked product obtained by cross-linking the polysaccharide having a carboxyl group and the cationic compound using a condensing agent or cross-linking agent.

3. The myocardial pad according to claim 2, wherein the condensing agent is water-soluble carbodiimide.

4. The myocardial pad according to claim 1, wherein the cross-linked product is a cross-linked product obtained by freeze-drying an aqueous solution containing the polysaccharide having a carboxyl group and the cationic compound, and then cross-linking the polysaccharide having a carboxyl group and the cationic compound using a condensing agent or cross-linking agent.

5. The myocardial pad according to claim 1, wherein the polysaccharide having a carboxyl group is glycosaminoglycan.

6. The myocardial pad according to claim 5, wherein the glycosaminoglycan is hyaluronic acid.

7. The myocardial pad according to claim 1, wherein the cationic compound is polylysine.

8. The myocardial pad according to claim 1, wherein a molar ratio between the polysaccharide having a carboxyl group and the cationic compound is 1:9 to 1:1.

9. The myocardial pad according to claim 1, which is used in cardioversion or pacing.

10. A myocardial lead comprising the myocardial pad according to claim 1 and a lead having an end attached to the pad.

11. A therapeutic apparatus for cardiac disease comprising the myocardial lead according to claim 10.

12. The therapeutic apparatus for cardiac disease according to claim 11, which is a defibrillator or cardiac pacemaker.
